(19) United States
(12) Patent Application Publication
(10) Pub. No.: US 2023/0309847 A1
(43) Pub. Date: Oct. 5, 2023

(54) APPARATUS AND METHOD FOR MEASURING TRIGLYCERIDE LEVEL

(71) Applicant: SAMSUNG ELECTRONICS CO., LTD., Suwon-si (KR)

(72) Inventors: Yun S PARK, Suwon-si (KR); Yeol Ho LEE, Suwon-si (KR); Joon Hyung LEE, Suwon-si (KR)

(73) Assignee: SAMSUNG ELECTRONICS CO., LTD., Suwon-si (KR)

(21) Appl. No.: 18/112,319

(22) Filed: Feb. 21, 2023

(30) Foreign Application Priority Data
Apr. 4, 2022 (KR) 10-2022-0041635
Jun. 17, 2022 (KR) 10-2022-0073991

(51) Int. Cl. A61B 5/0535; A61B 5/256; G16H 10/40; G16H 20/30; G16H 20/60

(52) U.S. Cl. CPC A61B 5/0535; A61B 5/256; G16H 10/40; G16H 20/30; G16H 20/60

(57) ABSTRACT

An apparatus for non-invasively measuring a triglyceride level based on bio-impedance and a method thereof are provided. The apparatus for measuring a triglyceride level according to an embodiment includes: an impedance sensor configured to measure bio-impedance of a user; and a processor configured to extract a bio-resistance value in a predetermined frequency band from the measured bio-impedance, to input user information and the extracted bio-resistance value to a learning model, and to measure a triglyceride level based on an output value of the learning model.

### CROSS-REFERENCE TO RELATED APPLICATION(S)

This application claims priority from Korean Patent Application No. 10-2022-0041635, filed on Apr. 4, 2022, and Korean Patent Application No. 10-2022-0073991, filed on Jun. 17, 2022 in the Korean Intellectual Property Office, the entire disclosure of which is herein incorporated by reference for all purposes.

### BACKGROUND

### 1. Field

Example embodiments of the disclosure relate to technology for non-invasively measuring triglyceride levels by using bio-impedance.

### 2. Description of the Related Art

Research on information technology (IT)-medical convergence technology, in which IT and medical technology are combined, is being recently carried out to address the aging population structure, rapid increase in medical expenses, and shortage of specialized medical service personnel. Particularly, monitoring of the health condition of the human body is not limited to a fixed place, such as a hospital, but is expanding to a mobile healthcare sector for monitoring a user’s health status at any time and any place in daily life at home and office.

### SUMMARY

In one general aspect, there is provided an apparatus for measuring a triglyceride level, the apparatus including: an impedance sensor configured to measure bio-impedance of a user; and a processor configured to extract a bio-resistance value in a predetermined frequency band from the measured bio-impedance, configured to input user information and the extracted bio-resistance value to a learning model, and configured to measure a triglyceride level based on an output value of the learning model.

The processor may obtain the user information, including at least one of age, gender, height, or weight, from a user via a user interface.

The processor may obtain the user information, including at least one of age, gender, height, or weight, from an application installed in the apparatus for measuring a triglyceride level or from an application installed in an external electronic device.

The predetermined frequency band may have a frequency of 5 kHz.

In addition, the apparatus for measuring a triglyceride level may further include a memory configured to store the learning model.

The learning model may include a non-linear machine learning model including support vector machine regression with radial basis function kernel (SVR-RBF).

The processor may output information for guiding the user to measure the triglyceride level at a predetermined time.

Based on the measured triglyceride level, the processor may provide the user with a health-related information including at least one of warning, diet information, or exercise information.

The processor may further collect health data including at least one of a blood pressure, a body mass index (BMI) score, an underlying condition, a type of exercise, an amount of exercise, an ingested food, or a triglyceride level measured at a previous time, and may provide the health-related information by using the measured triglyceride level and the collected health data.

In another general aspect, there is provided a method of measuring a triglyceride level by an apparatus for measuring a triglyceride level, the method including: by using an impedance sensor, measuring bio-impedance of a user; extracting a bio-resistance value in a predetermined frequency band from the measured bio-impedance; inputting user information and the extracted bio-resistance value to a learning model; and measuring a triglyceride level based on an output value of the learning model.

In addition, the method of measuring a triglyceride level may further include obtaining the user information, including at least one of age, gender, height, or weight, from a user via a user interface.

In addition, the method of measuring a triglyceride level may further include obtaining the user information, including at least one of age, gender, height, or weight, from another application installed in the apparatus for measuring a triglyceride level or in an external electronic device.

In this case, the predetermined frequency band may have a frequency of 5 kHz.

The learning model may include a non-linear machine learning model including support vector machine regression with radial basis function kernel (SVR-RBF).

In addition, the method of measuring a triglyceride level may further include, based on the measured triglyceride level, providing the user with health-related information including at least one of warning, diet information, or exercise information.

The providing of the health-related information may include collecting health data including at least one of a blood pressure, a body mass index (BMI) score, an underlying condition, a type of exercise, an amount of exercise, an ingested food, or a triglyceride level measured at a previous time, and providing the user with the health-related based on the measured triglyceride level and the collected health data.

In yet another general aspect, there is provided an electronic device including: a memory configured to store one or more instructions; and a processor, which by executing the one or more instructions, is configured to extract a bio-resistance value in a predetermined frequency band from bio-impedance of a user, to input user information and the extracted bio-resistance value to a learning model, and to measure a triglyceride level based on an output value of the learning model.

In addition, the electronic device may further include an output interface, which based on the measured triglyceride level, is configured to output a health-related information including at least one of warning, diet information, or exercise information.

In addition, the electronic device may further include an impedance sensor configured to measure the bio-impedance of the user.

In addition, the electronic device may further include a communication interface configured to receive the bio-impedance, measured by the impedance sensor, from another electronic device.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an apparatus for measuring a triglyceride level according to an embodiment of the present disclosure;

FIG. 2 is a block diagram illustrating an apparatus for measuring a triglyceride level according to another embodiment of the present disclosure;

FIGS. 3A to 3F are diagrams illustrating examples of inputting user information and providing health guidance by an apparatus for measuring a triglyceride level according to example embodiments;

FIG. 4 is a flowchart illustrating a method of measuring a triglyceride level according to an embodiment of the present disclosure; and

FIGS. 5 to 7 are diagrams illustrating examples of structures of an electronic device including an apparatus for measuring a triglyceride level according to an example embodiment.

### DETAILED DESCRIPTION

Details of other embodiments are included in the following detailed description and drawings. Advantages and features of the present invention, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as “unit” or “module”, etc., should be understood as a unit for performing at least one function or operation and that may be embodied as hardware, software, or a combination thereof.

Hereinafter, various embodiments of an apparatus and method for measuring a triglyceride level will be described with reference to the accompanying drawings. Various embodiments thereof may be included in an electronic device, such as a smartphone, a tablet PC, a desktop computer, a laptop computer, or a wearable device such as a wristwatch-type wearable device, a bracelet-type wearable device, a wristband-type wearable device, a ring-type wearable device, a glasses-type wearable device, an earphone-type wearable device, a necklace-type wearable device, an anklet-type wearable device, a headband-type wearable device, and the like.

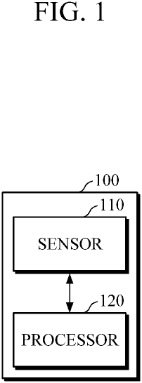

FIG. 1 is a block diagram illustrating an apparatus for measuring a triglyceride level according to an embodiment of the present disclosure.

Referring to FIG. 1, an apparatus 100 for measuring a triglyceride level includes a sensor 110 and a processor 120.

The sensor 110 may include an impedance sensor for measuring bio-impedance of a user. For example, the impedance sensor may include a pair of current electrodes and a pair of voltage electrodes to measure impedance by a four-electrode method. When a user’s skin is in contact with the current electrodes and the voltage electrodes, impedance may be measured by applying a current to the current electrodes and measuring a voltage using the pair of voltage electrodes; alternatively, impedance may be measured by applying a constant voltage to the pair of voltage electrodes and measuring a current flowing through the pair of current electrodes. However, the manner of impedance measurement is not limited thereto. For example, the impedance sensor may be configured to measure impedance by using a two-electrode method. The sensor 110 may further include a photoplethysmogram (PPG) sensor, an Electrocardiography (ECG) sensor, an Electromyography (EMG) sensor, etc., and may be formed as a single chip.

The processor 120 may be connected to the sensor 110 to control the sensor 110, and may receive sensor data from the sensor 110 and measure bio-information, such as triglycerides, skeletal muscle, fat mass, blood pressure, blood glucose, calories, skin carotenoid, blood carotenoid, glucose, urea, lactate, total protein, cholesterol, ethanol, vascular age, arterial stiffness, aortic pressure waveform, stress index, fatigue level, and the like.

For example, the processor 120 may measure a fasting triglyceride level based on bio-impedance measured at one or more frequencies, for example, in a frequency band of 1 kHz to 1 MHz by the impedance sensor. The processor 120 may extract a resistance value in a predetermined frequency band from the bio-impedance of a user, and may measure the triglyceride level by using the extracted resistance value. In this case, the predetermined frequency band may be 5 kHz. However, the frequency band is not limited thereto, and may be, for example, in a predetermined range having a frequency of 5 kHz, or may be a plurality of predefined frequency bands in the range of 1 kHz to 1 MHz.

The processor 120 may collect user information and may measure the triglyceride level by further using the collected user information in addition to the bio impedance resistance value. In this case, the user information may include, for example, age, gender, height, weight, smoking status, etc., but is not limited to the above example. For example, the processor 120 may provide a user interface so that a user may input the user information through the user interface, and may collect the user information from the user through the user interface. Alternatively, the processor 120 may also collect user information from a healthcare application installed in the apparatus 100 for measuring a triglyceride level or in another electronic device.

The processor 120 may measure the triglyceride level by using a learning model having, as input, the bio-impedance resistance value and the user information, e.g., age, gender, height, and weight, and having the triglyceride level as output. In this case, the learning model may be based on a linear and/or non-linear machine learning mapping function. For example, the machine learning mapping function may include Lasso regression, support vector machine regression (SVR), support vector machine regression with radial basis function kernel (SVR-RBF), etc., but is not limited thereto.

The processor 120 may measure the triglyceride level on-demand in response to a user’s request for measuring the triglyceride level. Alternatively, the processor 120 may guide a user (e.g., output information for guiding a user) to measure a fasting triglyceride level at a predetermined time, e.g., at a time when the user is in a fasting state (e.g., 7 a.m. every day). A measurement time of the triglyceride level may be set to one or more times every day. In this case, when the user carries or wears the apparatus 100 at the set time, such that the user is in a measurement state of the triglyceride level, such as in the case where the sensor 110 is in contact with the user’s skin, the processor 120 may automatically start measuring the triglyceride level instead of providing separate guidance (or health-related information). Alternatively, if the user is in a state in which measurement of the triglyceride level is possible, the processor 120 may continuously measure the triglyceride level during the entire duration in which triglyceride level measurement is possible or a portion of such duration, or at predetermined time intervals during the duration.

If predetermined conditions are met, the processor 120 may train the learning model again to calibrate the learning model. For example, the processor 120 may periodically calibrate the learning model. Alternatively, when user information is changed, the processor 120 may determine the change in user information automatically or in response to a user’s request, and may calibrate the learning model. For example, the processor 120 may determine that a user’s age is changed at the beginning of every year or at a time when the user’s birthday has passed, and may calibrate the learning model. In another example, the processor 120 may monitor a change in weight and the like based on health data of the user, and if the weight is changed to a predetermined threshold or more, the processor 120 may calibrate the learning model. In this case, the processor 120 may collect the health data through the user interface or from a healthcare application installed in the apparatus 100 or in another electronic device.

Upon determining to perform calibration, the processor 120 may control the sensor 110 to measure the bio-impedance of a user, and may collect user information. By using, as training data, the user information and the obtained bio-impedance at the calibration time, and/or user information and bio-impedance obtained at a previous calibration time or at a triglyceride level measurement time, or a measured triglyceride level, etc., the processor 120 may train the learning model again. In this manner, the processor 120 may build a learning model personalized for each user.

Upon measuring the triglyceride level, the processor 120 may generate health guidance information, such as warning, diet information, exercise information, etc., based on the measured triglyceride level, and may provide a user with the health guidance information by using various output means. For example, the processor 120 may determine whether the measured triglyceride level is normal (e.g., under 150 mg/dL), borderline (e.g., 150 mg/dL to 199 mg/dL), or high (200 mg/dL). Upon determining that the triglyceride level is normal, the processor 120 may inform a user of the normal level, and may guide (e.g., output information for guiding) the user to maintain the current diets or exercise. Alternatively, upon determining that the triglyceride level is at the borderline (e.g., 150 mg/dL to 199 mg/dL), or high (200 mg/dL), the processor 120 may provide warning information using an alarm, message, etc., in which case the processor 120 may provide the user with recommended diet or exercise which may be commonly applied.

The processor 120 may collect health data, such as a user’s diet data (e.g., ingested food, amount of food intake, number of times of food intake per day, etc.), exercise data (type of exercise, amount of exercise per day, etc.), and/or blood pressure, body mass index (BMI) score, underlying condition, previous measured triglyceride level, etc., through the user interface or from a healthcare application installed in the apparatus 100 or in another electronic device. The processor 120 may analyze the collected user information (e.g., height, age, weight, gender, etc.), the user’s diet information, exercise information, and/or health data, etc., and may provide the user with guidance, such as customized diet or exercise, etc., based on the analysis. For example, even when the current measured triglyceride level falls within the normal range, if there are factors that adversely affect the triglyceride level in the user’s current diet data, exercise data, and/or the health data, the processor 120 may guide the user to remove or reduce the factors. Further, if the triglyceride level is at the borderline level (e.g., 150 mg/dL to 199 mg/dL) or high level (200 mg/dL), the processor 120 may provide guidance by generating recommendations on customized diet or exercise for the user to reduce the triglyceride level.

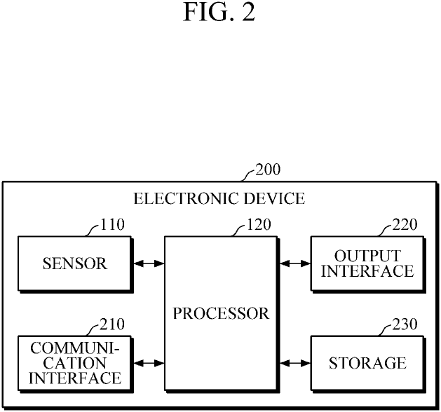

FIG. 2 is a block diagram illustrating an apparatus for measuring a triglyceride level according to another embodiment of the present disclosure.

Referring to FIG. 2, an apparatus 200 for measuring a triglyceride level includes the sensor 110, the processor 120, a communication interface 210, an output interface 220, and a storage 230. The sensor 110 and the processor 120 are described in detail above, and thus a description thereof will be omitted below.

The communication interface 210 may communicate with another electronic device under the control of the processor 120 by using communication techniques. The communication interface 210 may transmit sensor data measured by the sensor 110 and triglyceride level data generated and processed by the processor 120 to another electronic device. By using the installed healthcare application, the another electronic device may manage the triglyceride level data received from the apparatus 200 for measuring a triglyceride level, as well as data related to body composition information such as skeletal muscle mass, basal metabolic rate, body water, body fat percentage, etc., and/or exercise information such as step count, running distance, etc., and may provide the data to a user. In addition, the communication interface 210 may receive user information and data, such as a user’s health data, diet data, exercise data, etc., from the electronic device. Alternatively, the communication interface 210 may receive a learning model generated by the electronic device, or may receive a user’s bio-impedance measured by an impedance sensor of the electronic device. In this case, the processor 120 may measure a user’s triglyceride level by using the bio-impedance received from the electronic device through the communication interface 210.

The communication techniques used in the communication interface 210 may include, for example but not limited to, Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, mobile communication, etc., are not limited thereto.

The output interface 220 may output the sensor data measured by the sensor 110, the data generated and processed by the processor 120, and/or the data received through the communication interface 210. For example, the output interface 220 may output a user interface to a display so that a user may input a variety of information. Alternatively, the output interface 220 may output guidance information, including the measured triglyceride level, warning, diet, exercise, etc., which is generated by the processor 220, by using a display module, a speaker, a haptic device, and the like.

The storage 430 may store various instructions to be executed by the processor 120. In addition, the storage 230 may store data generated and/or processed by the sensor 110, the processor 120, the communication interface 210, etc., which may be referred to by the processor 120 during measurement of triglyceride levels. For example, the storage 230 may store health guidance information, such as a user’s diet data, exercise data, health data, recommended diet and exercise, etc., learning model, calibration conditions, and the like.

The storage 230 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

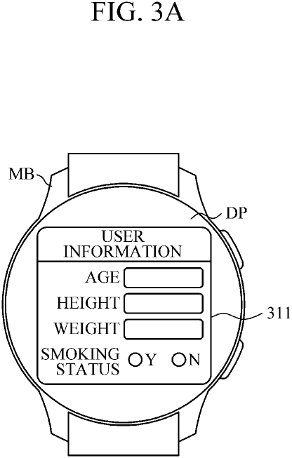

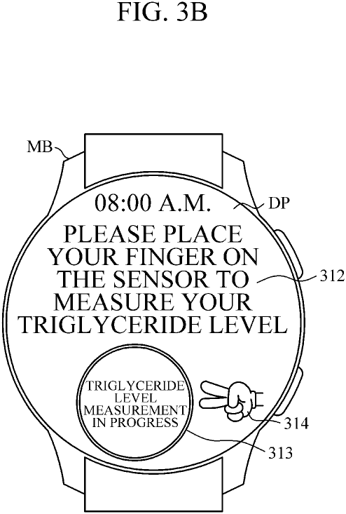

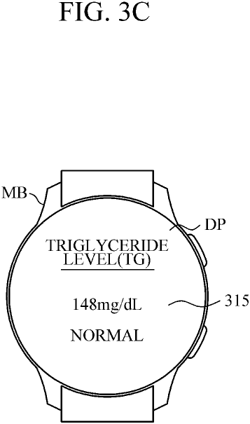

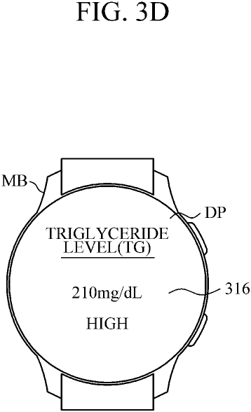

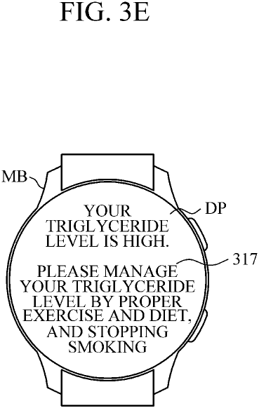

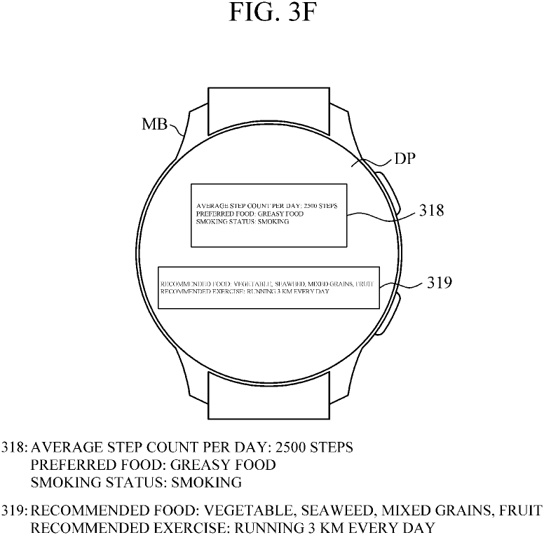

FIGS. 3A to 3F are diagrams illustrating examples of inputting user information and providing health guidance by an apparatus for measuring a triglyceride level according to example embodiments. For example, the examples described in FIGS. 3A to 3F may be provided by using the processor 120 of FIGS. 1 and 2. However, the following implementations are merely exemplary for convenience of explanation, and the present disclosure is not limited thereto.

Referring to FIG. 3A, the processor 120 may control the output interface 220 to output a user interface 311 to a display DP of a main body MB so that a user may input user information through the user interface 311. The user interface 311 may be provided for the user to input data, such as age, height, weight, smoking status, as well as various other data. The user interface 311 may be provided every time triglyceride levels are measured, in response to determination that the user information is changed, in response to a request for providing user information, and/or in response to receipt of user information from another electronic device, so that the user may change or update the information via the user interface 311.

Referring to FIG. 3B, the processor 120 may output, through the output interface 220, information 312 for guiding a user to measure a triglyceride level to the display DP of the main body MB. For example, as illustrated herein, the processor 120 may output a text, such as “please place your finger on the sensor to measure your triglyceride level,” and/or a sensor image 313 and a finger image 314 for guiding the user to place the finger on a sensor position. For example, the sensor image 313 may indicate a position of the sensor to which a user’s finger is to be placed, and the finger image 314 may point a direction toward the sensor.

Referring to FIGS. 3C and 3D, the processor 120 may output, through the output interface 220, a measured triglyceride level and/or graphic objects 315 and 316 (text, icon, image, etc.), indicating whether the triglyceride level is normal, to the display DP of the main body MB. In this case, based on whether the triglyceride level is normal, the processor 120 may output the graphic objects in different types and colors, so that the user may easily distinguish between normal and abnormal levels. In FIG. 3C, the graphic object 315 indicates that the triglyceride level is normal, along with a text that indicates the measured triglyceride level (i.e., 148 mg/dL), and in FIG. 3D, the graphic object 316 indicates that the triglyceride level is high, along with a text that indicates the measured triglyceride level (i.e., 210 mg/dL).

Referring to FIG. 3E, if the measured triglyceride level is abnormal, the processor 120 may output a warning text 317, such as “Your triglyceride level is high. Please manage your triglyceride level by proper exercise and diet, and stopping smoking”, and the like.

Referring to FIG. 3F, if the measured triglyceride level is abnormal, the processor 120 may output a text 318 for informing a user of the current diet data, exercise data, and/or health data, etc., and/or a text 319 for providing health guidance such as recommended food and exercise, and the like.

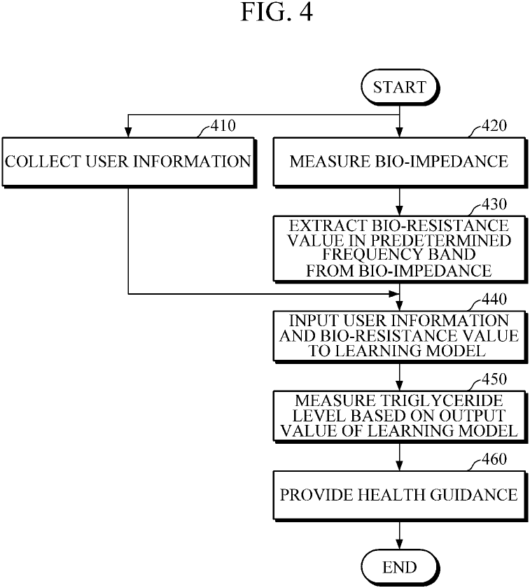

FIG. 4 is a flowchart illustrating a method of measuring a triglyceride level according to an embodiment of the present disclosure.

The method of FIG. 4 is an example of a method of measuring a triglyceride level which is performed by any one of the apparatus 100 and 200 for measuring a triglyceride level of FIGS. 1 and 2.

The apparatus for measuring a triglyceride level may collect user information, for example, gender, age, height, weight, and the like in 410. The apparatus for measuring a triglyceride level may receive the user information from a user through the user interface or may collect the user information from a healthcare application. It may not be required to perform operation 410 every time the triglyceride level is measured, and in an example embodiment, after being first performed once, operation 410 may be performed periodically, or in response to a user’s request or determination that user information is changed. In addition, operation 410 is not necessarily performed before measuring of bio-impedance in 420, and may be performed before operation 440.

Then, the apparatus for measuring a triglyceride level may measure bio-impedance of a user by using the impedance sensor in 420.

Subsequently, the apparatus for measuring a triglyceride level may extract a bio-resistance value in a predetermined frequency band from the bio-impedance in 430. In this case, the predetermined frequency band may be 5 kHz, but is not limited thereto.

Next, the apparatus for measuring a triglyceride level may input user information and the bio-resistance value to a learning model in 440, and may measure the triglyceride level based on an output value of the learning model in 450. The learning model may be a non-linear machine learning model for outputting the triglyceride level by using the user information and bio-resistance value as input.

Then, the apparatus for measuring a triglyceride level may provide health guidance based on the measured triglyceride level in 460. The health guidance may include the measured triglyceride level, information on whether the triglyceride level is normal, warning, diet, exercise, and the like.

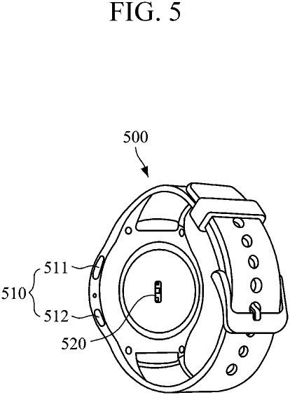

FIGS. 5 to 7 are diagrams illustrating examples of structures of an electronic device including an apparatus for measuring a triglyceride level according to an example embodiment. However, the present disclosure is not limited to the illustrated examples.

Referring to FIG. 5, the electronic device may be implemented as a wristwatch wearable device 500, and may include a main body and a wrist strap. A display is provided on a front surface of the main body, and may display various application screens including time information, received message information, estimated triglyceride level, and the like. As illustrated herein, an impedance sensor 510 may be disposed on a side surface of the main body. The impedance sensor 510 may include a first electrode part 511 and a second electrode part 512 which are spaced apart from each other to come into contact with two fingers respectively, and each of the first electrode part 511 and the second electrode part 512 may include a pair of electrodes.

In addition, the wearable device 500 may further include a sensor device 520 including a PPG sensor, a force sensor, etc., and disposed on a rear surface of the main body. When the main body is worn on a user’s wrist, the sensor device 520 may measure a PPG signal, a force signal, etc., at a region of the wrist. When the user wears the main body on the wrist of one hand and places a finger of the other hand on the impedance sensor 510, the sensor device 520 may measure the PPG signal and the like at the region of the wrist at the same time when the impedance sensor 510 measures bio-impedance.

A processor and various other components may be disposed in a main body case. The processor may obtain a triglyceride level by using the bio-impedance measured by the impedance sensor 510, and when the PPG signal and the like are measured by the sensor device 520, the processor may further obtain bio-information, such as blood pressure and the like, by using the PPG signal.

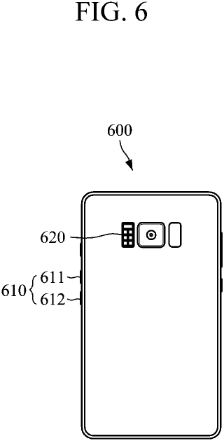

Referring to FIG. 6, the electronic device may be implemented as a mobile device 600 such as a smartphone.

The mobile device 600 may include a main body case and a display panel. The main body case may form an outer appearance of the mobile device 600. The main body case has a front surface, on which the display panel and a cover glass are disposed sequentially, and the display panel may be exposed to the outside through the cover glass. As illustrated herein, an impedance sensor 610, including a first electrode part 611 and a second electrode part 612, may be disposed on a side surface of the main body. In addition, a separate sensor device 620 for measuring a PPG signal, a force signal, and the like may be disposed on a rear surface of the main body. However, the arrangement is not limited thereto, and the impedance sensor 610 may be disposed near the sensor device 620 disposed on the rear surface of the main body; alternatively, the sensor device 620 may be disposed between the first electrode part 611 and the second electrode part 612 of the impedance sensor 610 or next to the impedance sensor 610.

A processor and various other components may be disposed in a main body case. The processor may obtain a triglyceride level by using the bio-impedance measured by the impedance sensor 610, and when the PPG signal and the like are measured by the sensor device 620, the processor may obtain bio-information, such as blood pressure and the like, by using the PPG signal.

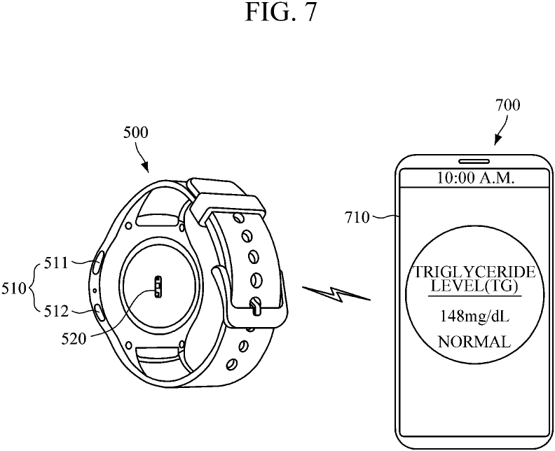

FIG. 7 is a diagram illustrating an example of obtaining a triglyceride level and providing health guidance by connecting the wearable device 500 with a mobile device 700. For example, a triglyceride level may be estimated by using a processor and the impedance sensor 510 of the wearable device 500, and the mobile device 700 may receive health guidance information as a result of the triglyceride level measurement (or triglyceride level estimation) from the wearable device 500, and may output the health guidance information to a display 710. In another example, bio-impedance may be measured by the impedance sensor 510 of the wearable device 500, and the mobile device 700 receives the bio-impedance data from the wearable device 500 and may obtain the triglyceride level based on the received bio-impedance data and output the obtained triglyceride level. The opposite case is also possible (that is, the wearable device 500 receive a triglyceride level and/or bio-impedance data from the mobile device 700 and, based thereon, outputs health guidance information).

The present disclosure may be provided based on a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium may be distributed over a plurality of computer systems connected to a network so that a computer-readable code is written thereto and executed therefrom in a decentralized manner. Functional programs, codes, and code segments needed for realizing the present invention may be readily deduced by programmers of ordinary skill in the art to which the invention pertains.

At least one of the components, elements, modules or units (collectively “components” in this paragraph) represented by a block in the drawings may be embodied as various numbers of hardware, software and/or firmware structures that execute respective functions described above, according to an example embodiment. According to example embodiments, at least one of these components may use a direct circuit structure, such as a memory, a processor, a logic circuit, a look-up table, etc. that may execute the respective functions through controls of one or more microprocessors or other control apparatuses. Also, at least one of these components may be specifically embodied by a module, a program, or a part of code, which contains one or more executable instructions for performing specified logic functions, and executed by one or more microprocessors or other control apparatuses. Further, at least one of these components may include or may be implemented by a processor such as a central processing unit (CPU) that performs the respective functions, a microprocessor, or the like. Two or more of these components may be combined into one single component which performs all operations or functions of the combined two or more components. Also, at least part of functions of at least one of these components may be performed by another of these components. Functional aspects of the above exemplary embodiments may be implemented in algorithms that execute on one or more processors. Furthermore, the components represented by a block or processing steps may employ any number of related art techniques for electronics configuration, signal processing and/or control, data processing and the like.

The present disclosure has been described herein with regard to example embodiments. However, it will be obvious to those skilled in the art that various changes and modifications may be made without changing technical conception and essential features of the present disclosure. Thus, it is clear that the above-described embodiments are illustrative in all aspects and are not intended to limit the present disclosure.

What is claimed is:

1. An apparatus for measuring a triglyceride level, the apparatus comprising:
an impedance sensor configured to measure bio-impedance of a user; and
a processor configured to extract a bio-resistance value in a predetermined frequency band from the measured bio-impedance, configured to input user information and the extracted bio-resistance value to a learning model, and configured to measure a triglyceride level based on an output value of the learning model.

2. The apparatus of claim 1, wherein the processor is configured to obtain the user information, including at least one of age, gender, height, or weight, from the user via a user interface.

3. The apparatus of claim 1, wherein the processor is configured to obtain the user information, including at least one of age, gender, height, or weight, from an application installed in the apparatus for measuring the triglyceride level or from an application installed in an external electronic device.

4. The apparatus of claim 1, wherein the predetermined frequency band has a frequency of 5 kHz.

5. The apparatus of claim 1, further comprising a memory configured to store the learning model.

6. The apparatus of claim 1, wherein the learning model comprises a non-linear machine learning model including support vector machine regression with radial basis function kernel (SVR-RBF).

7. The apparatus of claim 1, wherein the processor is configured to output information for guiding the user to measure the triglyceride level at a predetermined time.

8. The apparatus of claim 1, wherein based on the measured triglyceride level, the processor is configured to provide the user with health-related information including at least one of warning, diet information, or exercise information.

9. The apparatus of claim 8, wherein the processor is further configured to collect health-related data including at least one of a blood pressure, a body mass index (BMI) score, an underlying condition, a type of exercise, an amount of exercise, an ingested food, or a triglyceride level measured at a previous time, and provide the health-related information by using the measured triglyceride level and the collected health data.

10. A method of measuring a triglyceride level by an apparatus for measuring a triglyceride level, the method comprising:
by using an impedance sensor, measuring bio-impedance of a user;
extracting a bio-resistance value in a predetermined frequency band from the measured bio-impedance;
inputting user information and the extracted bio-resistance value to a learning model; and
measuring a triglyceride level based on an output value of the learning model.

11. The method of claim 10, further comprising obtaining the user information, including at least one of age, gender, height, or weight, from the user via a user interface.

12. The method of claim 10, further comprising obtaining the user information, including at least one of age, gender, height, or weight, from an application installed in the apparatus for measuring the triglyceride level or from an application an external electronic device.

13. The method of claim 10, wherein the predetermined frequency band has a frequency of 5 kHz.

14. The method of claim 10, wherein the learning model comprises a non-linear machine learning model including support vector machine regression with radial basis function kernel (SVR-RBF).

15. The method of claim 10, further comprising, based on the measured triglyceride level, providing the user with health-related information including at least one of warning, diet information, or exercise information.

16. The method of claim 15, wherein the providing the health-related information comprises collecting health data including at least one of a blood pressure, a body mass index (BMI) score, an underlying condition, a type of exercise, an amount of exercise, an ingested food, or a triglyceride level measured at a previous time, and providing the user with the health-related information based on the measured triglyceride level and the collected health data.

17. An electronic device comprising:
a memory configured to store one or more instructions; and
a processor configured to execute the one or more instructions, to extract a bio-resistance value in a predetermined frequency band from bio-impedance of a user, to input user information and the extracted bio-resistance value to a learning model, and to measure a triglyceride level based on an output value of the learning model.

18. The electronic device of claim 17, further comprising an output interface configured to, based on the measured triglyceride level, output a health-related information including at least one of warning, diet information, or exercise information.

19. The electronic device of claim 17, further comprising an impedance sensor configured to measure the bio-impedance of the user.

20. The electronic device of claim 19, further comprising a communication interface configured to receive the bio-impedance, measured by the impedance sensor, from another electronic device.
